(19) United States
(12) Patent Application Publication
(10) Pub. No.: US 2020/0046233 A1
(43) Pub. Date: Feb. 13, 2020

(54) PULSE WAVE EVALUATION APPARATUS AND PULSE WAVE EVALUATION METHOD

(71) Applicants: Kabushiki Kaisha Toshiba, Tokyo (JP); Toshiba Electronic Devices & Storage Corporation, Tokyo (JP)

(72) Inventor: Ken Kawakami, Kawasaki Kanagawa (JP)

(21) Appl. No.: 16/299,570

(22) Filed: Mar. 12, 2019

(30) Foreign Application Priority Data
Aug. 10, 2018 (JP) 2018-151981

(51) Int. Cl. A61B 5/021; A61B 5/00

(52) U.S. Cl. CPC A61B 5/02116; A61B 5/02416; A61B 5/7239

(57) ABSTRACT

A pulse wave evaluation apparatus has a measurer to measure a pulse wave of a subject, a time detector to detect, per one beat of the measured pulse wave, a rising time of the pulse wave, a time when a first-order differentiation value of the pulse wave with time becomes maximum, and a maximum amplitude time of the pulse wave, a ratio detector to detect an acceleration ratio of mean acceleration from the rising time to the time when the first-order differentiation value becomes maximum and mean acceleration from the time when the first-order differentiation value becomes maximum to the maximum amplitude time, and an evaluator to evaluate the pulse wave based on the acceleration ratio.

### CROSS REFERENCE TO RELATED APPLICATIONS

This application is based upon and claims the benefit of priority from the prior Japanese Patent Application No. 2018-151981, filed on Aug. 10, 2018, the entire contents of which are incorporated herein by reference.

### FIELD

Embodiments of the present disclosure relate to a pulse wave evaluation apparatus and a pulse wave evaluation method.

### BACKGROUND

A photoplethysmogram (PPG) sensor, which measures the change in blood volume in arteries and capillary vessels corresponding to the change in heart rate to detect pulse waves in accordance with heartbeat, has been known. A method of using the PPG sensor to detect the heart rate based on the blood volume passing through tissue per heart rate is referred to as a blood volume pulse (BVP) measurement.

The waveform of blood volume pulse largely varies depending on the active or metal state of a subject, so that the blood volume pulse becomes irregular. When the blood volume pulse is irregular, the heart rate and the like cannot be accurately measured. Especially, since the cardiac cycle depends on the differential coefficient of blood volume pulse from the blood-volume pulse rising time to the peak value time, if the blood volume pulse is irregular, the differential coefficient cannot be detected accurately, and hence the cardiac-cycle measurement accuracy is reduced.

### BRIEF DESCRIPTION OF THE DRAWINGS

FIG. 1 a block diagram schematically showing the configuration of a pulse wave evaluation apparatus according to an embodiment;

FIG. 2 is a figure showing an example of a wristwatch-type biometric measuring apparatus;

FIG. 3 is an example of the waveform of a normal pulse wave for one beat;

FIG. 4 is a figure showing variation distribution of a ratio AR;

FIG. 5 is a figure showing an example of a pulse wave of a subject;

FIG. 6 is a flowchart showing a process of the pulse wave evaluation apparatus according to the present embodiment;

FIG. 7A is a figure showing a result of the measurement of heart rate using pulse waves determined as normal; and

FIG. 7B is a figure showing a result of the measurement of heart rate without removing irregular pulse waves.

### DETAILED DESCRIPTION

According to one embodiment, a pulse wave evaluation apparatus comprising:

a measurer to measure a pulse wave of a subject;

a time detector to detect, per one beat of the measured pulse wave, a rising time of the pulse wave, a time when a first-order differentiation value of the pulse wave with time becomes maximum, and a maximum amplitude time of the pulse wave;

a ratio detector to detect an acceleration ratio of mean acceleration from the rising time to the time when the first-order differentiation value becomes maximum and mean acceleration from the time when the first-order differentiation value becomes maximum to the maximum amplitude time; and

an evaluator to evaluate the pulse wave based on the acceleration ratio.

Hereinafter, an embodiment will now be explained with reference to the accompanying drawings. In the following embodiment, a unique configuration and operation of a pulse wave evaluation apparatus will be mainly explained. However, the pulse wave evaluation apparatus may have other configurations and operations omitted in the following explanation.

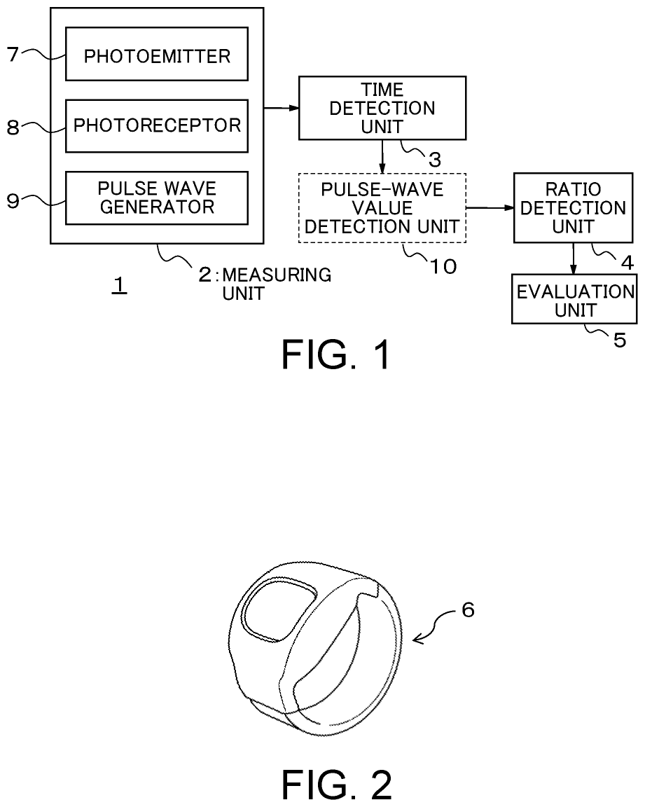

FIG. 1 is a block diagram schematically showing the configuration of a pulse wave evaluation apparatus 1 according to an embodiment. The pulse wave evaluation apparatus 1 is provided with a measuring unit (measurer) 2, a time detection unit (time detector) 3, a ratio detection unit (ratio detector) 4, and an evaluation unit (evaluator) 5. The pulse wave evaluation apparatus 1 may, for example, be built in a wristwatch-type biometric measuring apparatus 6 such as shown in FIG. 2.

The measuring unit 2 measures the change in blood volume of arteries and capillary vessels in accordance with the change in heart rate of a subject to acquire information on the blood volume pulse in accordance with heartbeat. Hereinafter, the blood volume pulse is simply referred to as a pulse wave as required.

The measuring unit 2 has a photoemitter 7, a photoreceptor 8, and a pulse wave generator 9. The photoemitter 7 has, for example, an LED (Light Emitting Diode) that emits an optical signal in a predetermined wavelength band (green, near-infrared band, etc.). The photoreceptor 8 receives a signal that is the optical signal from the photoemitter 7, after reflected and diffused in the body of a subject. The pulse wave generator 9 generates a pulse wave per one beat of heartbeat based on the optical signal received by the photoreceptor 8.

When the emission amount of the optical signal from the photoemitter 7 varies, the reception amount of the signal at the photoreceptor 8 also varies. For this reason, the pulse wave generator 9 separates the received optical signal into a D. C. component and an A. C. component, and generates a pulse wave based on the A. C./D. C. ratio. Therefore, the generated pulse wave is non-dimensional data.

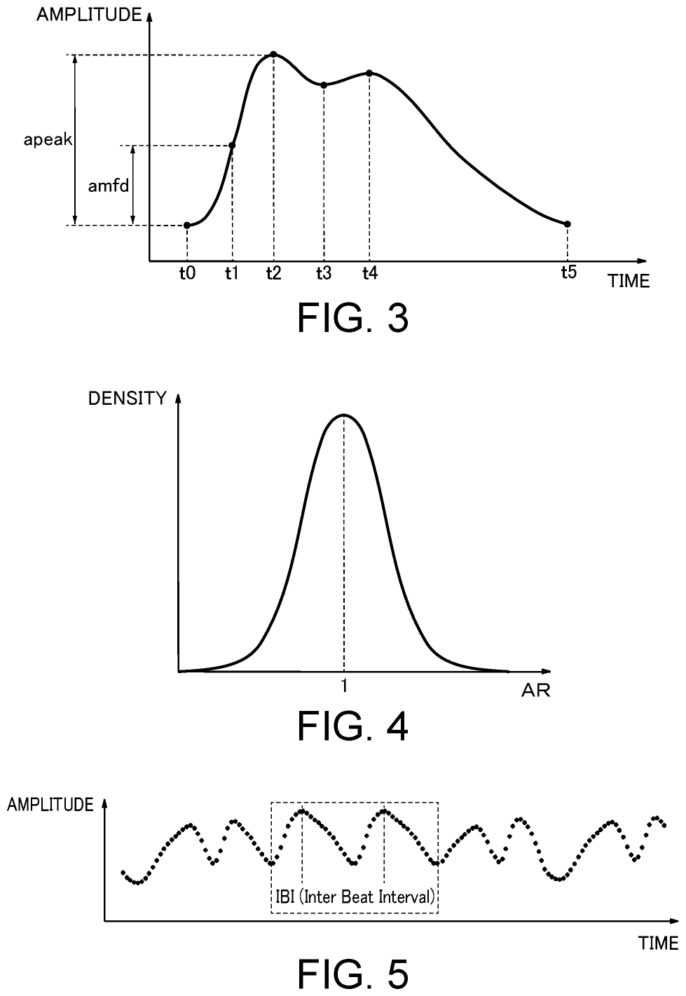

The time detection unit 3 detects, per one beat of the pulse wave, a rising time of the pulse wave, a time at which a value, which is obtained by differentiating the pulse wave with time by first-order differentiation, becomes maximum, and a time at which the amplitude of the pulse wave becomes a maximum peak. FIG. 3 is an example of the waveform of a normal pulse wave for one beat, the abscissa and ordinate indicating time and pulse wave amplitude, respectively. The normal pulse wave shows change in such a manner to begin at a position (t0) of the bottom of amplitude, reach a maximum amplitude peak (t2) with almost monotonic increase, thereafter, reach the second bottom of amplitude (t3) with monotonic decrease, reach the second amplitude peak (t4) again with monotonic increase, and reach the bottom value (t5) with monotonic decrease to complete.

The time detection unit 3 detects t0 of FIG. 3 as the rising time and detects t2 as the time at which the amplitude becomes the maximum peak. Moreover, the time detection unit 3 detects t1 that is the time at which the value, which is obtained by differentiating the pulse wave with time by first-order differentiation, becomes maximum, between t0 and t2.

The ratio detection unit 4 detects an acceleration ratio of mean acceleration of the pulse wave from the rising time t0 to the time t1 at which the value, which is obtained by differentiating the pulse wave with time by first-order differentiation, becomes maximum, and mean acceleration of the pulse wave from the time t1 to the maximum amplitude time t2.

The pulse wave evaluation apparatus 1 may be provided with a pulse-wave value detection unit 10. The pulse-wave value detection unit 10 detects a first value of the pulse wave at the maximum amplitude time t2 and a second value of the pulse wave at the time t1 at which the value obtained by first-order differentiation becomes maximum. The ratio detection unit 4 can detect the acceleration ratio based on a pulse-wave value ratio of the first and second values.

According to the examination of pulse waves having various waveforms by the present inventor, it is found that a normal pulse wave has an acceleration ratio of about 1 whereas an irregular pulse wave has an acceleration ratio having a value different from 1. Accordingly, the evaluation unit 5 evaluates the pulse wave based on the acceleration ratio detected by the ratio detection unit 4.

In more specifically, the evaluation unit 5 removes an irregular pulse wave to extract an unremoved pulse wave. The extracted pulse wave is used for the measurement of heart rate, the evaluation of blood circulation, etc.

Between mean acceleration a1 of the pulse wave between t0 to t1, an amplitude value x(t1) of the pulse wave at t1, and a first-order differential value dx(t1)/dt of the pulse wave to the time at t1, the following expression (1) is established.

$$2 {a}_{1} \cdot x ( {t}_{1} ) = {( \frac{dx ( {t}_{1} )}{dt} )}^{2} \qquad ( 1 )$$

In the same manner, between mean acceleration a2 of the pulse wave between t1 to t2, the amplitude value x(t1) of the pulse wave at t1, an amplitude value x(t2) of the pulse wave at t2, and the first-order differential value dx(t1)/dt at t1, the following expression (2) is established. A first-order differential value dx(t2) at t2 is dx(t2)=0.

$$2 {a}_{2} \cdot \{ x ( {t}_{2} ) - x ( {t}_{1} ) \} = - {( \frac{dx ( {t}_{1} )}{dt} )}^{2} \qquad ( 2 )$$

From the expressions (1) and (2), the following expression (3) is obtained.

2a1·x(t1)=2a2·{x(t1)−x(t2)} (3)

When the expression (3) is deformed, the ratio of mean acceleration of the pulse wave between t0 to t1 and t1 to t2 is expressed by the following expression (4).

$$\frac{{a}_{1}}{{a}_{2}} = 1 - \frac{x ( {t}_{2} )}{x ( {t}_{1} )} \qquad ( 4 )$$

From the above expression (4), an acceleration ratio AR of the mean acceleration between t0 to t1 and t1 to t2 is expressed by the following expression (5). Hereinafter, the acceleration ratio AR is simply referred to as a ratio AR as required.

$$AR = C 0 + C 1 \times \frac{{a}_{peak}}{{a}_{mfd}} \qquad ( 5 )$$

The term a_{peak is} the amplitude value x(t2) and the term a_{mfd} is the amplitude value x(t1). Coefficients C0 and C1 are any values, however, the coefficient C1 is set so that the mean value of AR becomes closer to 1. Or the coefficient C1 may be set under the condition of the coefficient C0=0.

The terms aₚₑₐₖ and a_{mfd} are differential data between two points of the pulse wave. By taking the ratio of aₚₑₐₖ and a_{mid}, the influence of variation in emission amount of the optical signal from the photoemitter 7 can be canceled.

Although it is not easy to directly detect the mean acceleration a1 and a2, it is relatively easy to detect the amplitude values x(t1) and x(t2). Therefore, in the present embodiment, instead of directly detecting the mean acceleration to obtain the acceleration ratio, the ratio AR is detected based on the expression (5).

As described above, the ratio detection unit 4 defines a value as the acceleration ratio, the value being obtained by multiplication of the pulse-wave value ratio between the pulse wave value aₚₑₐₖ at t2 and the pulse wave value a_{mfd} at t1 by the coefficient (first coefficient) C1 and by addition of the coefficient (second coefficient) C0 to a value obtained by the multiplication.

Since the ratio AR is different per one beat, the ratio AR per heartbeat is considered to vary in normal distribution as shown in FIG. 4. Population standard deviation of a normal distribution curve is relatively small, which is, for example, smaller than 0.15. In other words, once the coefficient C1 is decided, irrespective of the active state of a subject, it can be determined whether each pulse wave is irregular from a detected value of the ratio AR and estimated values of population mean and population standard deviation of the normal distribution curve.

A practical method of determining whether each pulse wave is irregular can, for example, be performed in the following procedure. It is required that the difference between a ratio Ari in an i-th pulse wave and a ratio ARi−1 in a (i−1)-th pulse wave is smaller than the coefficient C2.

|ARi−1−ARi|<C2 (6)

The expression (6) is established under the condition that pulse waves adjacent to each other have similar ratios AR.

Other than the above, it can be applied as a condition that the ratio AR is included in a predetermined range having the population mean as a criterion based on the assumption that the ratio AR varies in normal distribution. This condition is expressed by the following expression (7).

E(AR)−C3<ARi<E(AR)+C4 (7)

The term E(AR) is an estimated value of population mean of the ratio AR. Although, the coefficients C3 and C4 are any values, they may, for example, be ½ of an estimated value of population standard deviation.

For the coefficients C0 to C4 and E(AR), any initial values can be set, for example, initial setting may be performed such as C0=0, C1=0.5, E(AR)=1.06, C2=0.15, C3=0.15, and C4=0.15.

In this case, the expressions (5) to (7) are expressed by the following expressions (8) to (10), respectively.

ARi=aiₚₑₐₖ/(2.0×a_{imfd}) (8)

|ARi−1−ARi|<0.15 (9)

1.06−0.15<ARi<1.06+0.15 (10)

Pulse waves are stabilized so that ratios ARi of a larger number of pulse waves can satisfy the expressions (9) and (10). Thereafter, the population mean and population standard deviation of the detected ratio AR are estimated based on the assumption that the detected ratio AR varies in normal distribution. Using those estimated values, E(AR) and C0 to C4 are set, and, based the expressions (6) and (7), it may be more precisely determined whether the pulse wave is irregular per one beat. In other words, measurement of the pulse wave of a subject is started, and then, at the moment at which the expressions (9) and (10) are satisfied, it is determined that the pulse wave has become almost stable. Since, in the state where the pulse wave has become stable, it is considered that the pulse wave varies in normal distribution, the population mean and the population standard deviation can be obtained. Accordingly, the estimated value E(AR) of the population mean and the coefficients C0 to C4 in the expressions (6) and (7) can be obtained. Thereafter, based on the expressions (6) and (7), it is finally determined whether the pulse wave has become stable.

As described above, the evaluation unit 5 may estimate the population mean and population standard deviation of the normal distribution curve that indicates the degree of variation in acceleration ratio for a plurality of pulse waves measured by the measuring unit 2, and evaluate the pulse wave based on the estimated population mean and population standard deviation. In more specifically, the evaluation unit 5 may be provided with a first determiner to determine whether a difference in acceleration ratio for pulse waves adjacent to each other is smaller than a predetermined threshold value, a second determiner to determine whether the acceleration ratio is included in a predetermined range with the population mean as a criterion, and a determination evaluator to evaluate the pulse wave based on determination results of the first determination unit and the second determination unit. In this case, the determination evaluator determines that the pulse wave is irregular in the case where the first determiner has not determined that the difference in acceleration ratio is smaller than the predetermined threshold value or the second determiner unit has not determined that the acceleration ratio is included in the predetermined range.

FIG. 5 is a figure showing an example of a pulse wave of a subject. A waveform of the pulse wave of the subject changes in accordance with the active or mental state of the subject per one beat. The pulse wave evaluation apparatus 1 evaluates whether the pulse wave of the subject is irregular per one beat. In more specifically, it is determined whether the pulse wave satisfies the conditions of the above-described expressions (5) to (7) or (8) to (10), and, if so, it is determined that the pulse wave is normal with no irregular waves. In the example of FIG. 5, in the pulse wave of a plurality of beats, two heart beats enclosed by a broken-line frame are normal whereas the other heart beats are irregular. A normal pulse wave has a similar waveform to the waveform of FIG. 3. When two continuous pulse waves of two heart beats are normal, IBI (Inter-Beat Interval) can be calculated from the time between peak values of adjacent pulse waves and hence the heart rate and the like can be measured.

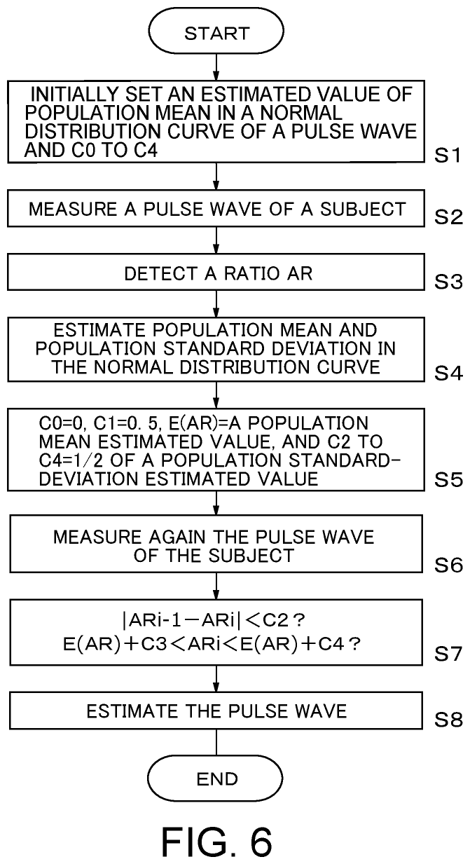

FIG. 6 is a flowchart showing a process of the pulse wave evaluation apparatus 1. Based on an assumption that the pulse wave of the subject varies in normal distribution, initial setting is made to the estimated value of population mean of the pulse wave and to the coefficients C0 to C4 (S1).

Subsequently, the pulse wave of the subject is measured by the measuring unit 2 (S2). Subsequently, to the waveform of the measured pulse wave, the ratio AR is detected based on the expression (5) (S3). The step S3 is performed per one beat of the pulse wave. Subsequently, the population mean and population standard deviation are estimated based on the values initially set in S1 and the ratio AR detected in S3 (S4).

Subsequently, setting is made such as C0=0, C1=0.5, E(AR)=a population-mean estimated value, and C2 to C4=½ of a population standard-deviation estimated value (S5). Or setting may be made such as C0=0, C1=0.5, E(AR)=1.06, and C2 to C4=0.15.

Subsequently, the pulse wave of the subject is measured again by the measuring unit 2 (S6). Then, it is determined whether the expressions (6) and (7) are satisfied (S7). Subsequently, the evaluation unit 5 evaluates the pulse wave based on a determination result in S7 (S8).

As for the way of evaluation in S8, a plurality of ways are considered. For example, a pulse wave that does not satisfy both of the expressions (6) and (7) may be removed as an irregular pulse wave. Or a pulse wave that does not satisfy the expression (6) may be removed as an irregular pulse wave. Or a pulse wave that does not satisfy the expression (7) may be removed as an irregular pulse wave.

Moreover, the determination as to whether the pulse wave is irregular may be made based on the expressions (6) and (7), under the condition that specific values are set such as E(AR)=1.06 and C2 to C4=0.07, without estimating the population mean and the population standard deviation.

As the flowchart of FIG. 6 shows, the evaluation unit 5 determines whether the pulse wave is irregular per one beat and extracts regular pulse waves only. When a subject is exercising hard, the pulse wave entirely becomes irregular largely, however, there are, for example, one or two regular pulse waves among several ten pulse waves. In the present embodiment, since regular pulse waves can be accurately extracted, by using the extracted normal pulse waves for the measurement of heart rate, blood circulation, etc., the measurement accuracy can be improved. Since, even if most pulse waves are irregular, if there are some regular pulse waves, various biometric information of the subject can be measured by using the regular pulse waves, so that according to the present embodiment, the biometric information of the subject can be measured accurately.

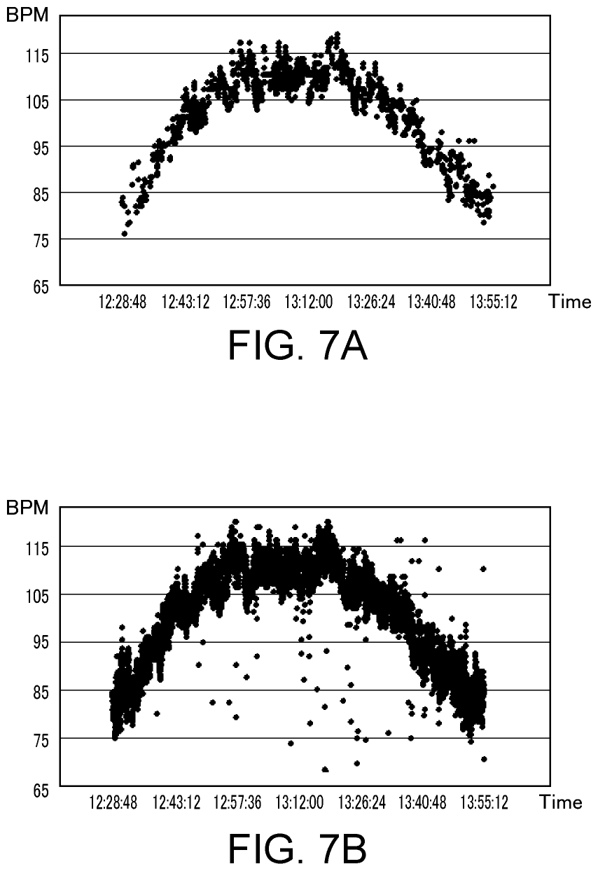

FIG. 7A is a figure showing a result of the measurement of heart rate using pulse waves determined as normal (not irregular) by the pulse wave evaluation apparatus 1. FIG. 7B is a figure showing a result of the measurement of heart rate without removing irregular pulse waves. In FIGS. 7A and 7B, the abscissa is heart-rate measuring time and the ordinate is heart rate (BPM: Beats Per Minute), plotting the heart rate at each time. The heart rate is a result of calculating 60,000/IBI per one beat.

In FIG. 7A, the heart rate shows small variation. By contrast, in FIG. 7B, the heart rate shows large variation, which shows that the heart-rate measurement accuracy is not good. In the case of FIG. 7B, since the pulse wave waveform varies, the time at which the first-order differential value is maximum cannot be correctly estimated. As a result, the mean acceleration value with this time as a criterion also varies, and hence the heart-rate measurement accuracy is reduced.

On the other hand, in the case of the present embodiment, as shown in FIG. 7A, the variation in heart rate can be restricted. In FIG. 7A, E(AR) is 1.06 based on the assumption of normal distribution, C1 is 2.0, and C2 to C4 are 0.07 obtained by dividing the population standard-deviation estimated value by 2.

As described above, in the present embodiment, the pulse wave is evaluated based on the acceleration ratio of the mean acceleration from the pulse-wave rising time to the time at which the value obtained by first-order differentiation becomes maximum, and the mean acceleration from the time at which the value obtained by first-order differentiation becomes maximum to the maximum amplitude time, during the period from the pulse-wave rising time to the maximum amplitude time. Therefore, whether the pulse wave is irregular can be determined easily and accurately per one beat.

Moreover, in the present embodiment, the ratio of mean acceleration is detected using a ratio of the pulse wave value at the maximum amplitude time and the pulse wave value at the time at which the value obtained by first-order differentiation becomes maximum. Therefore, the ratio can be relatively easily calculated, so that the pulse wave per one beat can be evaluated in real time.

According to the present embodiment, since a normal pulse wave can be extracted, the heart rate, cardiac cycle, etc. can be accuracy measured irrespective of the active or metal state of the subject.

At least part of the pulse wave evaluation apparatus 1 described above may be configured with hardware or software. When it is configured with software, a program that performs at least part of the pulse wave evaluation apparatus 1 may be stored in a storage medium such as a flexible disk and CD-ROM, and then installed in a computer to run thereon. The storage medium may not be limited to a detachable one such as a magnetic disk and an optical disk but may be a standalone type such as a hard disk and a memory.

Moreover, a program that achieves the function of at least part of the pulse wave evaluation apparatus 1 may be distributed via a communication network a (including wireless communication) such as the Internet. The program may also be distributed via an online network such as the Internet or a wireless network, or stored in a storage medium and distributed under the condition that the program is encrypted, modulated or compressed.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. A pulse wave evaluation apparatus comprising:
a measurer to measure a pulse wave of a subject;
a time detector to detect, per one beat of the measured pulse wave, a rising time of the pulse wave, a time when a first-order differentiation value of the pulse wave with time becomes maximum, and a maximum amplitude time of the pulse wave;
a ratio detector to detect an acceleration ratio of mean acceleration from the rising time to the time when the first-order differentiation value becomes maximum and mean acceleration from the time when the first-order differentiation value becomes maximum to the maximum amplitude time; and
an evaluator to evaluate the pulse wave based on the acceleration ratio.

2. The pulse wave evaluation apparatus of claim 1 further comprising a pulse-wave value detector to detect a first value of the pulse wave at the maximum amplitude time and a second value of the pulse wave at the time when the first-order differentiation value becomes maximum,
wherein the ratio detector detects the acceleration ratio based on a pulse-wave value ratio of the first and second values.

3. The pulse wave evaluation apparatus of claim 2, wherein the ratio detector defines a value as the acceleration ratio, the value being obtained by multiplication of the pulse-wave value ratio of the first value and the second value by a first coefficient and by addition of a second coefficient to a value obtained by the multiplication.

4. The pulse wave evaluation apparatus of claim 1, wherein the measurer measures a plurality of pulse waves of the subject, and
the evaluator estimates population mean and population standard deviation of a normal distribution curve indicating a degree of variation in the acceleration ratio for the plurality of pulse waves measured by the measurer, and evaluates the pulse wave based on the estimated population mean and population standard deviation.

5. The pulse wave evaluation apparatus of claim 4, wherein the evaluator comprises:
a first determiner to determine whether a difference in the acceleration ratio for two pulse waves adjacent to each other is smaller than a predetermined threshold value;
a second determiner to determine whether the acceleration ratio is included in a predetermined range with the population mean as a criterion; and
a determination evaluator to evaluate the pulse wave based on determination results of the first determiner and the second determiner.

6. The pulse wave evaluation apparatus of claim 5, wherein the evaluator determines that the pulse wave is irregular if the first determiner does not determine that the difference in the acceleration ratio is smaller than the predetermined threshold value or the second determiner dose not determine that the acceleration ratio is included in the predetermined range.

7. The pulse wave evaluation apparatus of claim 5, wherein the evaluator determines that the pulse wave is stable if the first determiner determines that the difference in the acceleration ratio is smaller than the predetermined threshold value and the second determiner determines that the acceleration ratio is included in the predetermined range.

8. A pulse wave evaluation method to be executed on computer comprising:
measuring a pulse wave of a subject;
detecting, per one beat of the measured pulse wave, a rising time of the pulse wave, a time when a first-order differentiation value of the pulse wave with time becomes maximum, and a maximum amplitude time of the pulse wave;
detecting an acceleration ratio of mean acceleration from the rising time to the time when the first-order differentiation value becomes maximum and mean acceleration from the time when the first-order differentiation value becomes maximum to the maximum amplitude time; and
evaluating the pulse wave based on the acceleration ratio.

9. The pulse wave evaluation method of claim 8 further comprising:
detecting a first value of the pulse wave at the maximum amplitude time and a second value of the pulse wave at the time when the first-order differentiation value becomes maximum; and
detecting the acceleration ratio based on a pulse-wave value ratio of the first and second values.

10. The pulse wave evaluation method of claim 9, wherein a value, obtained by multiplication of the pulse-wave value ratio of the first value and the second value by a first coefficient and by addition of a second coefficient to a value obtained by the multiplication, is defined as the acceleration ratio.

11. The pulse wave evaluation method of claim 8 further comprising:
measuring a plurality of pulse waves of a subject;
estimating population mean and population standard deviation of a normal distribution curve indicating a degree of variation in the acceleration ratio for the plurality of measured pulse waves; and
estimating the pulse wave based on the estimated population mean and population standard deviation.

12. The pulse wave evaluation method of claim 11 further comprising:
determining by a first determiner whether a difference in the acceleration ratio for two pulse waves adjacent to each other is smaller than a predetermined threshold value;
determining by a second determiner whether the acceleration ratio is included in a predetermined range with the population mean as a criterion; and
evaluating the pulse wave based on determination results of the first determiner and the second determiner.

13. The pulse wave evaluation method of claim 12, wherein it is determined that the pulse wave is irregular if the first determiner does not determine that the difference in the acceleration ratio is smaller than the predetermined threshold value or the second determiner dose not determine that the acceleration ratio is included in the predetermined range.

14. The pulse wave evaluation method of claim 12, wherein it is determined that the pulse wave is stable if the first determiner determines that the difference in the acceleration ratio is smaller than the predetermined threshold value and the second determiner determines that the acceleration ratio is included in the predetermined range.
